(19) United States
(12) Patent Application Publication
(10) Pub. No.: US 2009/0024047 A1
(43) Pub. Date: Jan. 22, 2009

(54) DEVICES AND METHODS FOR RESPIRATION THERAPY

(75) Inventors: Robert D. Shipley, Woodbury, MN (US); Rodney W. Salo, Fridley, MN (US)

Correspondence Address: PAULY, DEVRIES SMITH & DEFFNER, L.L.C., PLAZA VII- SUITE 3000, 45 SOUTH SEVENTH STREET, MINNEAPOLIS, MN, 55402-1630, US

(73) Assignee: CARDIAC PACEMAKERS, INC., St. Paul, MN (US)

(21) Appl. No.: 11/780,861

(22) Filed: Jul. 20, 2007

(51) Int. Cl. A61B 5/08; A61B 5/00; A61F 2/02

(52) U.S. Cl. 600/532; 600/301; 623/11.11; 705/3

(57) ABSTRACT

Embodiments of the invention are related to devices and methods for respiration therapy, amongst other things. In an embodiment, the invention includes a system for providing respiration therapy to a patient, including an implantable device comprising a chronically implanted respiration sensor, the respiration sensor configured to generate a signal indicative of respiration rate of the patient; and an external interface device in communication with the implantable device, the external interface device comprising an output device and configured to deliver respiration therapy to the patient, the respiration therapy comprising one or more breathing prompts generated by the output device. In an embodiment, the invention includes a method for providing respiration therapy to a patient. In an embodiment, the invention includes a method of monitoring a heart failure patient for decompensation events. Other aspects and embodiments are provided herein.

### TECHNICAL FIELD

This disclosure relates generally to devices and methods for respiration therapy and, more particularly, to devices and methods for modulating breathing characteristics of a patient, amongst other things.

### BACKGROUND OF THE INVENTION

The act of breathing, as part of the overall process of respiration, is one of the most fundamental homeostatic mechanisms of the body. The core function of breathing, of course, is transporting oxygen into the lungs and transporting carbon dioxide out of the lungs. However, because of various effects such as changes in intrathoracic pressure and nervous stimulation, breathing can affect other aspects of the body such as cardiac function. Therefore breathing and its characteristics such as respiration rate and tidal volume can impact various disease states.

One example of a disease state that can be affected by breathing characteristics is heart failure. According to the American Heart Association, nearly 5 million Americans are living with heart failure, and 550,000 new cases are diagnosed each year. Heart failure is a serious clinical syndrome in which an abnormality of cardiac function causes cardiac output to fall below a level adequate to meet the metabolic demand of peripheral tissues. Reduced cardiac output has significant negative effects including a depressing effect on renal function due to decreased renal perfusion, which causes increased fluid retention by the kidneys. The increased fluid retention by the kidneys results in an increased blood volume and further increased venous return to the heart, thus increasing the heart's preload. Increased fluid retention causes the progressive peripheral and pulmonary edema that characterizes overt congestive heart failure. As part of a downward spiral, diastolic filling pressure becomes further elevated which causes the heart to become so dilated and edematous that its pumping function deteriorates even more.

Some data suggests that the signs and symptoms of both systolic and diastolic heart failure can be improved through breathing modulation with techniques including repetitive prayer, yoga, tai chi and the like. This is because respiration rate, which is a result of both voluntary and involuntary control, can affect cardiac function and neurohumoral control which are disturbed with heart failure. In general, it has been found that heart failure patients derive benefits from a slowing of their respiration rate, as part of a multidisciplinary intervention strategy.

Some care providers have begun prescribing a therapeutic regimen that includes modification of patient's breathing habits. Such a regimen can be referred to as breathing therapy or respiration therapy. However, it can be difficult for some patients to comply with instructions regarding such therapy outside of the clinical setting. As such, patient compliance with the prescribed regimen is a concern. In addition, it is generally difficult for both patients and care providers to track progress in modulating breathing habits. In addition to the preventative therapeutic effect of breathing therapy, it has also been shown that acute breathing therapy can reduce the neurohumoral impact of acute decompensation and assist the patient in the emergency room.

For at least these reasons, a need exists for devices and methods for providing respiration therapy to patients.

### SUMMARY OF THE INVENTION

Embodiments of the invention are related to devices and methods for providing respiration therapy to patients, amongst other things. In an embodiment, the invention includes a system for providing respiration therapy to a patient including an implantable device comprising a chronically implanted respiration sensor. The respiration sensor can be configured to generate a signal indicative of respiration rate of the patient. The system can include an interface device in communication with the implantable device. The interface device can include an output device and be configured to deliver respiration therapy to the patient. The respiration therapy can include one or more breathing prompts generated by the output device.

In an embodiment, the invention includes a method for providing respiration therapy to a patient. The method can include transmitting a respiration signal of the patient from an implanted device to an external interface device. The method can also include delivering respiration therapy to the patient via an external interface device. The respiration therapy can include one or more prompts. The prompts can direct the patient to reduce their respiration rate to a rate less than or equal to a target rate.

In an embodiment, the invention includes a method of monitoring a heart failure patient for decompensation events. The method can include generating a respiration signal with a chronically implanted respiration sensor and monitoring the respiration signal for acute increases in respiration rate. The method can also include evaluating other physiological parameters of the patient and summoning emergency assistance if there is an acute increase in respiration rate and the other physiological parameters suggest an acute decompensation event is occurring. In some embodiments, the method can also include delivering respiration therapy to the patient via an external interface device in the event of an acute decompensation event.

In an embodiment, the invention includes an implantable system for providing respiration therapy to a patient. The system can include an implantable device comprising a chronically implanted respiration sensor, the respiration sensor configured to generate a signal indicative of respiration rate of the patient. The system can also include an electrical stimulation lead comprising an electrode, the electrical stimulation lead in electrical communication with the implantable device. The implantable device can be configured to administer respiration therapy to the patient, the respiration therapy including one or more breathing prompts, the breathing prompts including electrical stimulation pulses delivered to the phrenic nerve.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope of the present invention is defined by the appended claims and their legal equivalents.

### BRIEF DESCRIPTION OF THE DRAWINGS

The invention may be more completely understood in connection with the following drawings, in which:

FIG. 1 is a schematic view of a system for providing breathing modulation therapy in accordance with an embodiment of the invention.

FIG. 2 is a schematic view of an implantable device in accordance with an embodiment of the invention.

FIG. 3 is a schematic diagram of some components of an exemplary controller in accordance with an embodiment of the invention.

FIG. 4 is a schematic view of an implantable device in accordance with an embodiment of the invention.

FIG. 5 is a schematic view of a system for providing breathing modulation therapy in accordance with another embodiment of the invention.

FIG. 6 is a schematic view of a system for providing breathing modulation therapy in accordance with another embodiment of the invention.

FIG. 7 is a schematic view of one exemplary method of administering respiration therapy to a patient.

FIG. 8 is a graph of a target respiration rate over time in accordance with both a linear change approach and a non-linear change approach to providing respiration therapy.

FIG. 9 is a flow chart illustrating a method in accordance with an embodiment of the invention.

FIG. 10 is a flow chart illustrating a method in accordance with another embodiment of the invention.

FIG. 11 is a flow chart illustrating a method in accordance with another embodiment of the invention.

FIG. 12 is a flow chart illustrating a method in accordance with another embodiment of the invention.

While the invention is susceptible to various modifications and alternative forms, specifics thereof have been shown by way of example and drawings, and will be described in detail. It should be understood, however, that the invention is not limited to the particular embodiments described. On the contrary, the intention is to cover modifications, equivalents, and alternatives falling within the spirit and scope of the invention.

### DETAILED DESCRIPTION OF THE INVENTION

Breathing habits, such as respiration rate, can affect various disease states. For example, some data suggest that the symptoms of heart failure can be improved through modulation of breathing habits. As such, some care providers have begun prescribing a therapeutic regimen that includes modification of their breathing habits. However, it can be difficult for patients to comply with instructions regarding their breathing habits outside of the clinical setting. In addition, it is generally difficult for care providers and patients to track progress in modulating breathing habits.

However, embodiments of devices and methods as described herein can be used to provide breathing modulation therapy to patients in a highly automated way that, in some embodiments, can increase compliance, aid in monitoring compliance, and/or track the progress of patients. In a particular embodiment, the invention includes a system for providing breathing modulation therapy to a patient, including an implantable device comprising a respiration sensor, the respiration sensor configured to generate a signal indicative of respiration rate of the patient. The system also includes an external interface device, configured to generate one or more breathing prompts receivable by the patient. Embodiments of the invention also include methods related to the delivery of respiration therapy. Various aspects of embodiments will now be described in greater detail.

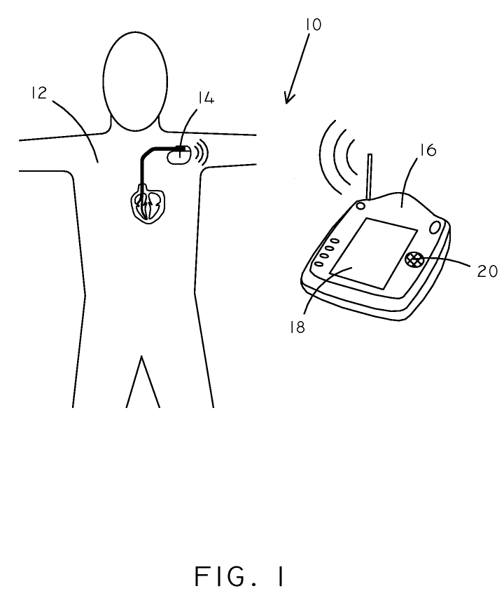

FIG. 1 is a schematic view of a system for providing respiration therapy in accordance with an embodiment of the invention. The system 10 includes an implantable device 14 capable of monitoring respiration and providing a signal representative of respiration, implanted within the body 12 of a patient. In some embodiments, the implantable device 14 can be an implantable cardiac rhythm management (CRM) device. By way of example, the device can be a pacemaker, a cardiac resynchronization therapy (CRT) device, a remodeling control therapy (RCT) device, a cardioverter/defibrillator, or a pacemaker-cardioverter/defibrillator.

The system 10 also includes an external interface device 16. The external interface device 16 can include a video output 18 and/or an audio output 20. The external interface device 16 can communicate with the implantable device 14 wirelessly. The external interface device 16 can take on many different forms. In some embodiments, the external interface device 16 can include a patient management system. An exemplary patient management system is the LATITUDE® patient management system, commercially available from Boston Scientific Corporation, Natick, Mass. Aspects of an exemplary patient management system are described in U.S. Pat. No. 6,978,182, the contents of which are herein incorporated by reference.

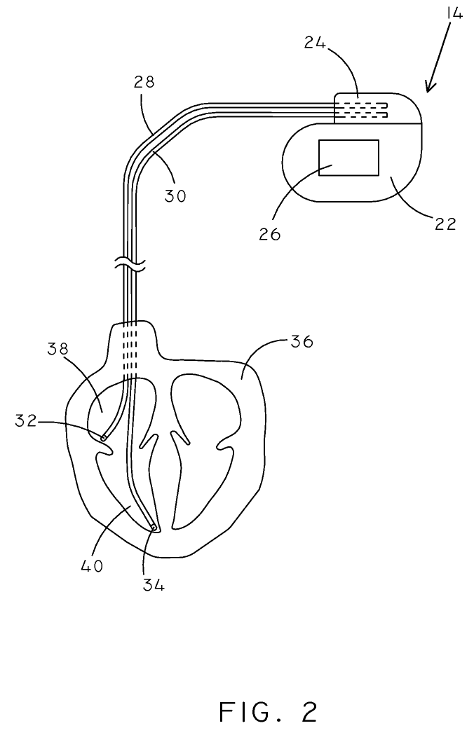

The implantable device 14 can include many different components. Referring now to FIG. 2, a schematic view of the implantable device 14 is shown. The implantable device includes a housing 22 and a header 24. The housing 22 can include a hermetically sealed chamber. Various circuitry components, such as a controller 26, can be disposed within the housing 22. One or more stimulation leads 28, 30 can be coupled to elements within the housing 22 through the header 24. One or more electrodes 32, 34 can be disposed on the stimulation leads 28, 30. The electrodes 32, 34 can be configured to engage cardiac tissues and deliver electrical stimulation pulses to the tissues. The electrodes can be in electrical communication with elements inside the housing via conductors disposed within the stimulation leads. The electrodes 32, 34, can be disposed within or around various parts of the heart 36, such as within the right atrium 38 or within the right ventricle 40. In some embodiments, a stimulation lead can be positioned so that one or more electrodes are disposed within the coronary venous system.

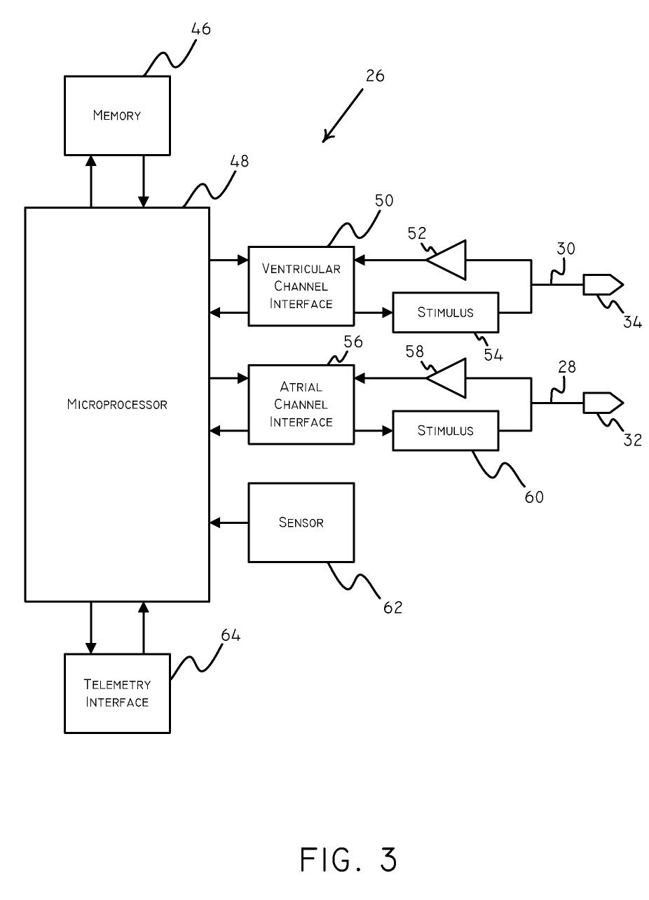

The controller 26 can include various electronic components and can be configured to perform operations and methods as described herein. Referring now to FIG. 3, a schematic diagram is shown of some components of an exemplary controller 26 and associated components. The controller 26 can include a microprocessor 48. The microprocessor 48 can execute instructions and can communicate with a memory 46 via a bidirectional data bus. The memory 46 typically comprises a ROM or RAM for program storage and a RAM for data storage. The controller can include one or more ventricular sensing and pacing channels including sensing amplifier 52, output circuit 54, and ventricular channel interface 50, which can be in communication with electrode 34 and stimulation lead 30. The controller can also include one or more atrial sensing and pacing channels including sensing amplifier 58, output circuit 60, and an atrial channel interface 56, which can be in communication with electrode 32 and stimulation lead 28. Both the ventricular sensing and pacing channels and the atrial sensing and pacing channels can communicate bidirectionally with a port of microprocessor 48. For each channel, the same stimulation lead and electrode can be used for both sensing and pacing.

The channel interfaces 50 and 56 can include analog-to-digital converters for digitizing sensing signal inputs from the sensing amplifiers and registers which can be written to by the microprocessor in order to output pacing pulses, change the pacing pulse amplitude, and adjust the gain and threshold values for the sensing amplifiers. The controller 26 can also interface with one or more sensors 62, such as an accelerometer, a posture sensor, an impedance sensor, a minute ventilation sensor, a pressure sensor, or the like. The controller 26 can also interface with a telemetry module 64 for communicating with an external interface device.

Implantable devices used with embodiments herein can include one or more respiration sensors configured to produce signals indicative of a patient's breathing. For example, a signal can be produced that is indicative of a patient's respiration rate. In some embodiments, the respiration sensor is an impedance sensor. For example, referring back to FIG. 2, the device can be configured to measure the impedance between electrode 34 and the housing 22, and/or the impedance between electrode 32 and the housing 22. There are many usable techniques for measuring impedance through bodily tissue. For example, one usable technique is described in Published U.S. Patent Application 2004/0102712, which is incorporated herein by reference in its entirety. Generally, a current is provided from electrode (such as 32 or 34) that travels through the body tissue to housing 22. Simultaneously, the voltage differential between housing 22 and electrode (32 or 34) is monitored. Based on the relationship between current and voltage, the impedance of the body tissue can be determined.

Parameters of respiration can then be derived by processing the respiration signal produced by the respiration sensor. For example, respiration rate can be derived by processing a signal from an impedance sensor. One exemplary technique for determining respiratory parameters such as minute ventilation, tidal volume, and respiratory rate based on an impedance signal is described in U.S. Pat. No. 6,275,727, the content of which is herein incorporated by reference. However, it will be appreciated that there are many other techniques for determining respiratory parameters based on the signal from an impedance sensor.

Respiration sensors can include other types of sensors beyond impedance sensors. For example, in some embodiments, the respiration sensor can include an accelerometer or a pressure sensor. Signals from accelerometers and pressure sensors will include fluctuations caused by respiration. For example, the signal from an accelerometer will fluctuate based on movements of the chest during respiration. As such, these signals can be processed in order to derive parameters of respiration such as respiration rate. Accelerometers used herein can include single axis accelerometers and multiple-axis accelerometers. An exemplary accelerometer is described in U.S. Pat. No. 6,937,900, the content of which is herein incorporated by reference. Pressure sensors used herein can include any type of pressure sensor, for example an electrical, mechanical, or optical pressure sensor, which generates a signal in response to pressure. By way of example, exemplary pressure sensors are described in U.S. Pat. No. 6,237,398, the content of which is herein incorporated by reference.

Embodiments of the invention can also include sensors configured to measure physiological parameters other than respiration parameters. Specifically, embodiments of the invention can include sensors configured to detect other physiological parameters of a patient that can be used to aid in the diagnosis of the patient's condition. For example, embodiments of the invention can include a pressure sensor disposed within the body in order to measure physiological pressures. As a specific example, a pressure sensor can be disposed within the venous system to measure venous pressure. As another example, a pressure sensor can be disposed within the pulmonary artery. Pressure sensors can also be disposed within the intrapleural space to measure pleural pressure. Many different measures of physiological condition can be derived from pressure sensors. By way of example, the contractions of the heart cause variations in physiological pressures and, as such, pressure signals can be processed in order to generate information regarding the functioning of the heart.

Embodiments of the invention can also include sensors configured to detect whether or not fluid is being retained by a patient. Generally, when excess fluid it being retained, osmolality of bodily fluids is reduced. As such, in some embodiments, systems described herein can include an osmolality sensor.

Embodiments of the invention can also include sensors to detect various parameters indicative of respiration such as arterial and/or venous concentrations of dissolved gases, such as oxygen and/or carbon dioxide. Sensors for dissolved gases in the blood are known to those of skill in the art. One example of an exemplary oxygen sensor is described in U.S. Pat. No. 4,815,469, the context of which related to oxygen sensors is herein incorporated by reference. It will be appreciated that similar devices can also be used to measure concentrations of dissolved carbon dioxide in the blood. It will be appreciated that many other sensors can also be used to detect dissolved gases in the blood.

Embodiments of the invention can include sensors configured to detect electrical activity within the body, such as the electrical activity of the heart. Signals from such sensors can be processed in order to derive information regarding functioning of the heart such as heart rate, heart rhythm, waveforms, and the like.

Components of systems herein, such as implanted medical devices and sensors, can be chronically implanted. The term “chronically implanted” as used herein with respect to a medical device shall refer to those medical devices that are implanted within an organism that are intended to remain implanted long-term, such as for a period of time lasting for months or years.

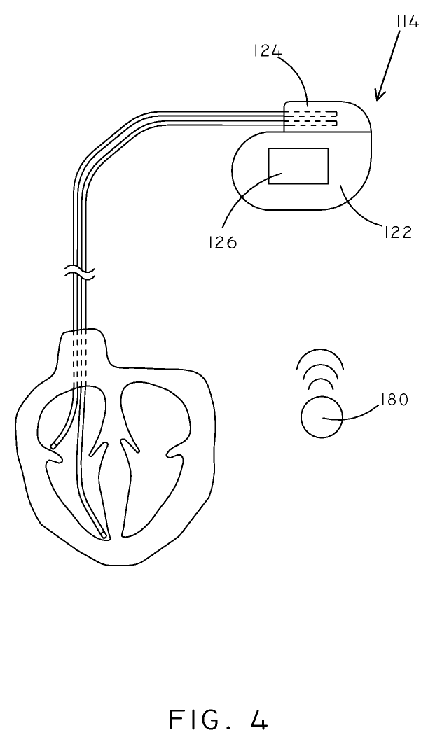

In some embodiments, sensors used can be coupled to or tethered to the implantable device. In other embodiments, sensors can be located remotely from the implantable device and can be configured to be in wireless communication with the implantable device. Referring now to FIG. 4, a schematic view of the implantable device 114 is shown in accordance with an embodiment of the invention. The implantable device includes a housing 122 and a header 124. The housing 122 can include a hermetically sealed chamber. Various circuitry, such as a controller 126, can be disposed within the housing 122. A sensor 180, such as a respiration sensor, can be disposed remotely from the other components of the implantable device 114. The sensor 180 can be in wireless communication with components within the housing 122. For example communication can take place acoustically, via radiofrequency transmission, inductively, etc.

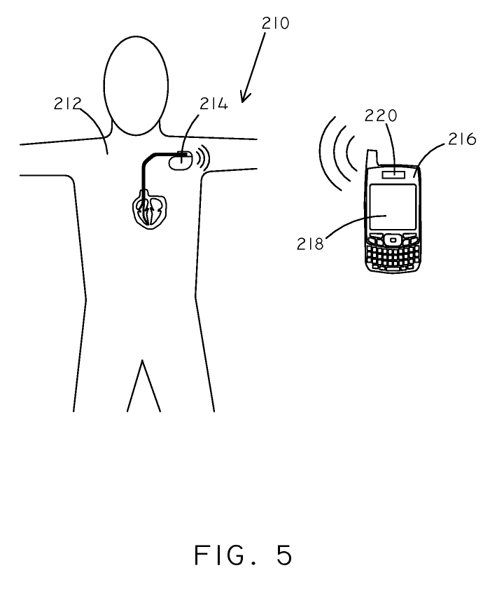

It will be appreciated that external interface devices used with embodiments of the invention can take on various forms. In some embodiments, the external interface device can be a handheld computing device with the ability to send and/or receive data wirelessly, such as a smart phone. Referring now to FIG. 5, a schematic view of a system for providing breathing modulation therapy is shown in accordance with another embodiment of the invention. The system 210 includes an implantable device 214, implanted with the body 212 of a patient. The system 210 also includes an external interface device 216. The external interface device 216 includes a video output 218 or an audio output 220. The external interface device 216 can communicate with the implantable device 214 wirelessly. The external interface device 216 can be a handheld computer device capable of wireless data transmission. For example, the external interface device 216 can be a smart phone or a handheld personal digital assistant.

In some embodiments, the external interface device can be small enough to be worn on the wrist of a patient, like a wrist watch. In embodiments where the external interface device is in close proximity to the skin of the patient, the external interface device and the implanted device can be in wireless communication using acoustic techniques, such as ultrasound technology. Well not intending to be bound by theory, it is believed that acoustic techniques can be advantageous because of their energy efficiency.

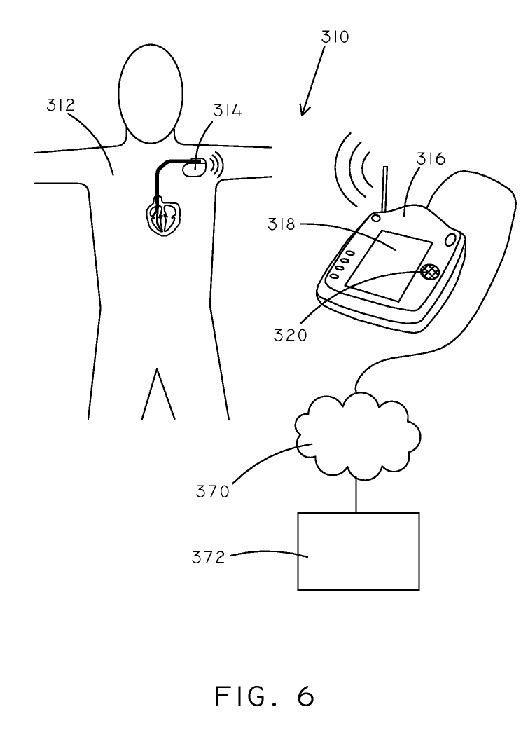

Some embodiments of devices herein can communicate through a data network in order to send or receive data to or from other points on the network. By way of example, some embodiments of device herein can be configured to send data regarding a patient's respiratory parameters to a care provider and this data can be communicated through a secured data network via the Internet. As another example, some embodiments of devices herein can communicate through a data network in order to send or receive alerts and/or to summon emergency assistance. Referring now to FIG. 6, a schematic view is shown of a system for providing breathing modulation therapy in accordance with an embodiment of the invention. The system 310 includes an implantable device 314, implanted with the body 312 of a patient. The system 310 also includes an external interface device 316. The external interface device 316 includes a video output 318 and an audio output 320. The external interface device 316 can communicate with the implantable device 314 wirelessly. The external interface device 316 can be in communication with a network 370, such as the Internet or a phone network. A care provider 372 can receive data from the external interface device 316 through the network 370. In addition, the care provider 372 can send data to the external interface device 316, such as operating instructions or queries through the network 370.

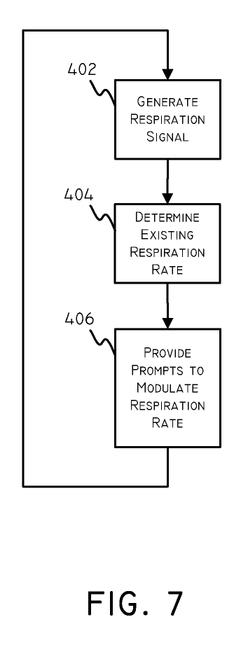

Respiration therapy can be administered to a patient with systems of the invention in many different ways. FIG. 7 is a schematic view of one exemplary method of administering respiration therapy to a patient. In operation 402, a respiration signal is generated. For example, a respiration signal can be generated by a respiration sensor as described herein. In operation 404, the existing respiration rate of the patient is determined. This can involve processing the respiration signal. In operation 406, prompts are provided in order to modulate the respiration rate. This can include providing prompts to gradually reduce the respiration rate if the existing respiration rate of the patient exceeds a desired level. It is also possible to provide prompts to encourage a patient to breath at a therapeutic rate without first determining the patient's respiration rate in various embodiments.

Some patients may have difficulty making sudden changes in their breathing habits, such as their respiration rate. As such, the system can be configured to provide prompts in order to gradually change the patient's breathing habits in order to reach a desired rate or range of rates. For example, if a given patient is initially breathing at a rate of 15 breaths per minute when a respiration therapy session first begins and a care provider has set a target respiration rate of 6 breaths per minute, then the system can be configured to gradually reduce the patient's respiration rate instead of suddenly reducing the respiration rate. In some embodiments, the gradual reduction can be implemented as a function of time either linearly or non-linearly. For example, in the case where the breathing rate is initially at a rate of 15 breaths per minute, then approximately four seconds elapses during each respiration cycle. This means that in order to hit a target respiration rate of 6 breaths per minute, the time for each cycle will have to be increased from four seconds up to ten seconds. In some embodiments, this change is effectuated linearly and in other embodiments non-linearly, such as with a step function.

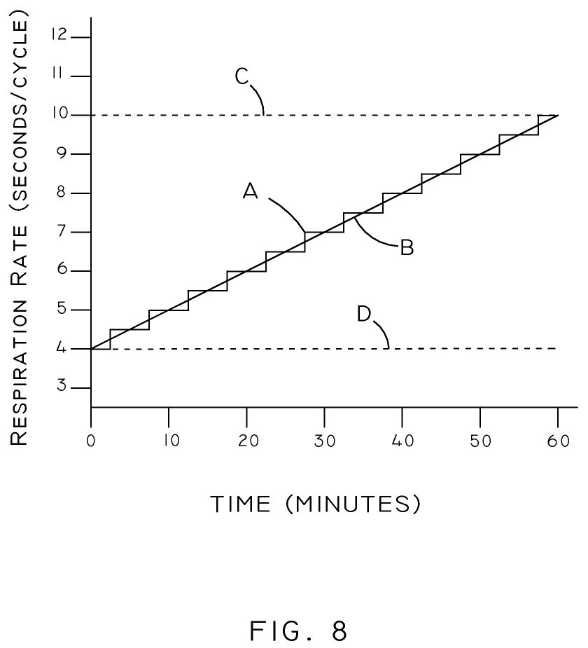

Referring now to FIG. 8, a graph is shown of target respiration rate over time in accordance with both a gradual linear change approach and a non-linear change approach to administering respiration therapy. First, a patient's preexisting respiration rate is determined. In this example, the preexisting respiration rate is approximately 4 seconds per cycle. The preexisting respiration rate is illustrated by line D in FIG. 8. In one approach to providing respiration rate therapy, the target respiration rate changes linearly over time to reach a therapeutic respiration rate. Linear change of the target respiration rate is illustrated by line B in FIG. 8. The therapeutic respiration rate is illustrated by line C in FIG. 8. Alternatively, a non-linear approach can be used to change the target respiration rate over time. For example, the change in the target respiration rate over time can follow a step function. Non-linear change of the target respiration rate is illustrated by line A in FIG. 8.

It will be appreciated that there are many different methods of changing the target respiration rate over time. For example, in some embodiments, the therapeutic respiration rate is arrived at relatively quickly, such as during less than half the time of the therapy session, and then the therapeutic respiration rate is maintained for the remainder of the session. In other embodiments, the therapeutic respiration rate is arrived at only by the end of the therapy session, such as is illustrated in FIG. 8.

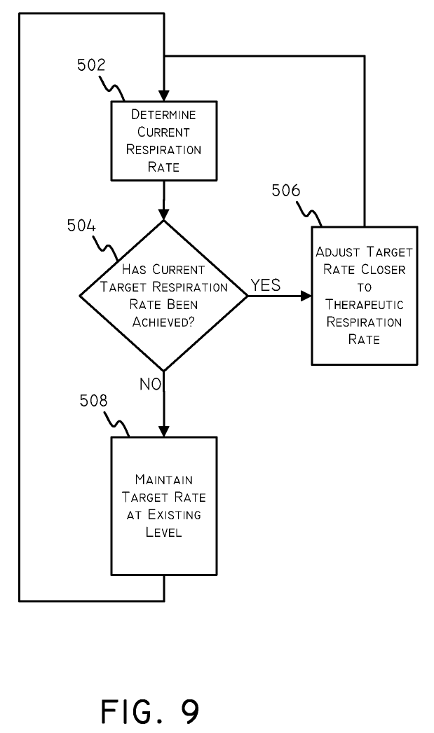

Another approach to changing the target respiration rate is illustrated in FIG. 9. In this method, feedback regarding the patient's current respiration rate is used in determining how to adjust the current target respiration rate. In operation 502, the patient's current respiration rate is determined. In operation 504, the current respiration rate is compared with the current target respiration rate. If the current target respiration rate has been achieved by the patient, then in operation 506 the current target respiration rate is adjusted to be incrementally closer to the therapeutic respiration rate desired, before starting over with operation 502. However, if the current target respiration rate has not yet been achieved by the patient, then in operation 508 the current target respiration rate is maintained at its current level, before starting over with operation 502.

Prompts given to patients in order to provide respiration therapy can include various directions including prompts to inhale, prompts to exhale, prompts to hold their breath, etc. These prompts can come in various forms including video prompts, audio prompts, tactile prompts, and the like. In some embodiments, when audio prompts are given, the prompts can include a voice stating what action the patient is supposed to be performing at a given time point. In other embodiments, when audio prompts are given, the prompts can include tones or rhythms that correspond to actions to be taken by the patient. When video prompts are given to a patient, such as through a video display, the prompts can include words, colors, numbers, or combinations of these in order to indicate what action the patient is supposed to be performing at given time points. In some embodiments, a countdown clock can be displayed in order to provide the patient with information regarding how long they are to perform the current action.

Prompts given to patients in order to provide respiration therapy can also include electrical stimulation pulses. For example, in some embodiments, such as where the system is a CRT device, electrodes on the coronary venous lead can be used to electrically stimulate the phrenic nerve providing a respiratory prompt for the patient. Exemplary techniques of delivering an electrical stimulation pulse to the phrenic nerve are described in U.S. Pat. No. 6,415,183, the content of which is herein incorporated by reference. In some embodiments, the same electrodes used for stimulation of cardiac tissue can be used for stimulation of the phrenic nerve. In other embodiments, separate electrodes are used for stimulation of cardiac tissue and stimulation of the phrenic nerve. Stimulation of the phrenic nerve at a sufficiently low level will provide a respiratory prompt without actually initiating diaphragmatic contraction. The correct stimulus location, amplitude and duration to provide such a respiratory prompt can be determined empirically at the time of implant.

In general, electrical stimulation of the phrenic nerve sufficient for a respiratory prompt will also stimulate the cardiac tissue and therefore it is generally necessary for respiratory prompt stimulation to be coordinated with cardiac stimulation. In embodiments where separate electrodes are used for stimulation of cardiac tissue and stimulation of the phrenic nerve, the respiratory prompt stimulation pulse and the cardiac stimulation pulse can be applied at a different frequency. For example, an inhalation prompt stimulation pulse and the cardiac stimulation pulse can be applied simultaneously, followed by three cardiac stimulation pulses without an accompanying respiratory prompt stimulation pulse. Then, an expiration prompt stimulation pulse and the cardiac stimulation pulse can be applied simultaneously, followed by five cardiac stimulation pulses without an accompanying respiratory prompt stimulation pulse. Then, the cycle can be repeated. In the context of electrical stimulation prompts, different types of prompts can be indicated by stimulation pulses with a greater amplitude or duration. For example, an inhalation prompt stimulation pulse can be differentiated from an expiration prompt stimulation pulse by a greater amplitude or duration.

In embodiments where the same electrodes are used for stimulation of cardiac tissue and stimulation of the phrenic nerve, a similar coordinated pattern of cardiac stimulation and breathing prompts can be applied. In such an embodiment, a higher energy pulse can be applied when both stimulation of cardiac tissue and a respiratory prompt are desired while a lower energy pulse can be applied when only stimulation of cardiac tissue is desired. Thus, in one exemplary cycle, a higher energy pulse can be administered followed by a number of lower energy pulses, before the cycle repeats.

Different types of instructions can be given to a patient as is desired based on the patient's condition, aptitude, preferences of the care provider, etc. For example, some patients may find it easiest to simply be provided with two prompts, one to signal inhalation and the other to signal exhalation. Other patients may find it beneficial to hold their breath for a period of time after inhalation when trying to slow down their respiration rate. As such, in some embodiments, patients can receive at least three different types of prompts including one prompt to signal inhalation, one prompt to signal exhalation, and one prompt to signal that they should hold their breath.

The sequence of different types of prompts can be manipulated as is desired for a given patient. For example, for some patients it may be desirable to insert a “hold breath” prompt after each inhalation and exhalation prompt. For other patients, it may be desirable to only provide them with a “hold breath” prompt after inhalation. Table 1 below illustrates some exemplary breathing prompt sequences. However, it will be appreciated that many other breathing prompt sequences are contemplated herein.

TABLE 1

| | Breathing Prompt Sequences | | |
|---|---|---|---|
| Cycle Number | Sequence 1 | Sequence 2 | Sequence 3 |
| Cycle 1 | Inhale, | Inhale, | Inhale, |
| | Exhale | Hold | Hold |
| | | Breath, | Breath, |
| | | Exhale | Exhale, |
| | | | Hold |
| | | | Breath |
| Cycle 2 | Inhale, | Inhale, | Inhale, |
| | Exhale | Hold | Hold |
| | | Breath, | Breath, |
| | | Exhale | Exhale, |
| | | | Hold |
| | | | Breath |
| Cycle 3 | Inhale, | Inhale, | Inhale, |
| | Exhale | Hold | Hold |
| | | Breath, | Breath, |
| | | Exhale | Exhale, |
| | | | Hold |
| | | | Breath |
| Cycle n | . . . | . . . | . . . |

The relative timing of different prompts corresponding to different phases of the respiratory cycle (e.g., inhalation, exhalation, hold breathing, etc.) can be configured as appropriate for the specific patient. By way of illustration, in the context of a breathing prompt sequence that contains only prompts to inhale and to exhale, the relative timing of the two prompts can be configured as desired. For example, the inhalation prompt could be displayed for 50% of the time and the exhalation prompt could be displayed for 50% of the time. Where the current target for a breathing cycle is 8 seconds, this could involve providing the inhalation prompt for 4 seconds and providing the exhalation prompt for 4 seconds. However, in normal breathing patterns, expiration generally takes longer than inspiration. As such, in some embodiments, the expiration prompt is displayed for a longer period of time than the inspiration prompt.

While not intending to be bound by theory, it is believed that some cardiac performance and neurohumoral benefits can be derived through breathing that is synchronized with cardiac contractions represented, for example, by electrocardiography. In general, synchrony for a dual oscillator system (such as the breathing cycle and the cardiac contraction cycle) can be described by the following equation:

|nφ₁−mφ₂|<ε (Equation 1)

wherein n and m are integers that describe the ratio of the synchronized oscillations, φ_{1,2} are phases of the oscillators, and ε is a small positive integer. In some embodiments of the invention, prompts delivered to patients are timed to promote synchrony between the breathing cycle and the cardiac contraction cycle, such as myocardial systole or diastole. Such synchronous prompting can take on many different forms. For example, in an embodiment, prompts to a patient to begin inhalation can be timed to coincide with the beginning of a cardiac contraction cycle or can be timed to coincide with the R wave of a electrogram (R waves correspond to contraction of the ventricles).

Respiration therapy can be administered for a period of time which can be referred to as a respiration therapy session. The respiration therapy session can last for any period of time desirable. The session length can be a parameter that is configured by a care provider. In some embodiments, the length of the respiration therapy session can be determined, in part, by the breathing performance of the patient using the system. By way of example, in some embodiments the respiration therapy session can be configured to end at some defined time point after a therapeutic respiration rate has been achieved. In an embodiment, the respiration therapy session can be configured to end approximately one half hour after a therapeutic respiration rate has been achieved by the patient.

In some embodiments, the therapeutic respiration rate is equal to or less than about 10 breaths per minute. In some embodiments, the therapeutic respiration rate is equal to about 6 breaths per minute. In some embodiments, the therapeutic respiration rate is equal to or less than about 8 breaths per minute. In some embodiments, the therapeutic respiration rate is equal to or less than about 6 breaths per minute.

In some embodiments, respiration therapy sessions can be initiated on a preselected periodic basis. For example, a care provider can program the system in order to initiate a respiration therapy session every day at a certain time. As another example, a care provider can program the system to initiate a respiration therapy session every other day during a preselected window of time. In some embodiments, respiration therapy sessions can be initiated by the patient.

In other embodiments, the system can be configured to monitor the breathing of a patient and initiate a respiration therapy session in response to adverse changes in the breathing pattern of the patient. Adverse changes can include an increase in a patient's respiration rate that is not coincident with an increase in heart rate. As such, the device can be configured to intervene when necessary and attempt to aid the patient by initiating a respiration therapy session.

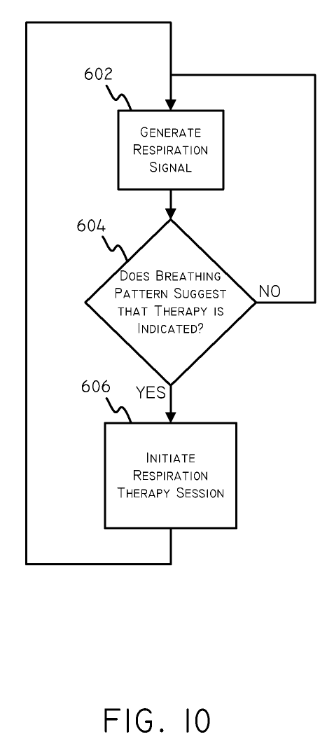

Referring now to FIG. 10, a flow chart is shown of a method whereby the system can initiate respiration therapy when necessary. In operation 602, the system can generate a respiration signal. By way of example, a respiration sensor can be used to generate a respiration signal. In operation 604, the system can evaluate the respiration signal in order to determine whether or not the breathing pattern of the patient suggests that respiration therapy is indicated and/or is safe. This can be done in various ways. As a first example, the respiration rate can simply be evaluated to see if it exceeds a predetermined threshold level. The threshold level can be programmed into the system by a care provider. As a second example, the current respiration rate can be compared with data regarding historical respiration rates for the patients. As an illustration, if some measure of the historical data is exceeded, such as the standard deviation of respiration rate data for the last ten days, then respiration therapy can be deemed to be indicated. As yet another example, the data regarding the current respiration rate can be evaluated in conjunction with data from one or more different types of sensors. As an illustration, a respiration rate signal can be evaluated in conjunction with data from an activity sensor (such as an accelerometer) or a posture sensor. In some embodiments, respiration therapy can be deemed to be indicated if the respiration sensor shows a significantly increased respiration rate where data from the other sensors suggest that this is not expected. By way of example, if data from the activity sensor suggests that the patient is inactive, but the respiration sensor shows a sharply elevated respiration rate, then respiration therapy can be deemed to be indicated.

In operation 606, if evaluation of the data in operation 604 suggests that a respiration therapy session is indicated, then the device can initiate a respiration therapy session. In some embodiments, the system can also send an alert to a care provider describing the patient's condition. In this manner, if a patient's overall breathing functions are declining and the system is repetitively initiating respiration therapy, the care provider will be alerted to this fact and is able to decide whether to call the patient into a care facility or query the device remotely in order to gather more information on the health status of the patient.

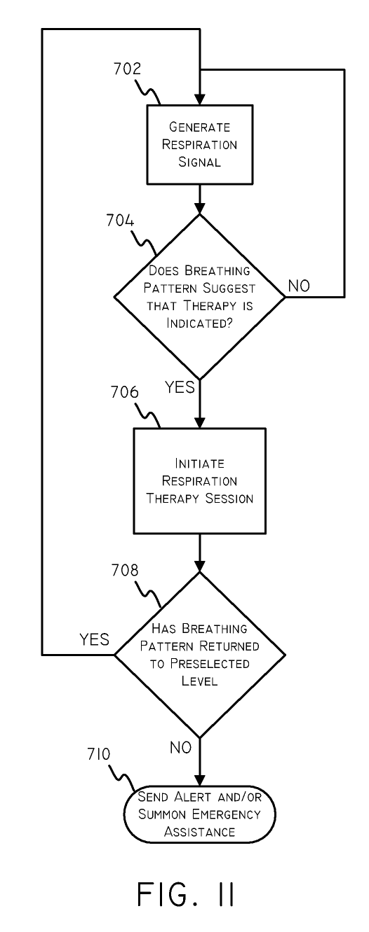

In some embodiments, the system can send an alert and/or summon emergency assistance if the patient's respiration signal does not return to predetermined values after the system delivers respiration therapy. By way of example, the system can be configured to send an emergency notification to a care provider noting the conditions of the patient. Referring now to FIG. 11, a flow chart is shown illustrating some aspects of a method in accordance with an embodiment of the invention. In operation 702, the system can generate a respiration signal. The respiration signal can be processed in order to derive information about a patient's breathing patterns, including the respiration rate. In operation 704, the system can evaluate whether respiration therapy is indicated. This can be done in various ways, such as by determining whether the respiration rate exceeds a threshold value. If respiration therapy is not indicated, then the system can go back to operation 702. However, if respiration therapy is indicated, then the system can initiate a respiration therapy session in operation 706. In operation 708, the system can evaluate whether or not the patient's breathing pattern has returned to some preselected level. If it has, then the system can go back to operation 702. If the patient's breathing pattern has not returned to a preselected level, then in operation 710 the system can send an alert notification and/or summon emergency assistance.

In some embodiments, when the system sends an alert or summons emergency assistance, the system can also include information regarding the current location of the patient. For example, the system can include GPS (global positioning system) functionality so that the position of the patient can be determined without input from the patient. In this manner, emergency assistance can be summoned without regard to the ability of the patient to provide necessary information.

In some embodiments, the system can monitor a patient for other symptoms that may aid in determining why the patient's breathing patterns have changed, such as why the patient's respiration rate has increased. By way of example, in the context of a heart failure patient, the system can be configured to monitor for other signs of acute decompensation, beyond just an increased respiration rate. Signs of acute heart failure can include fluid retention, tachycardia, elevated venous pressure, and changes in heart sounds, amongst others. In some embodiments, the system can be configured to detect signs of acute decompensation and send an alert and/or summon emergency assistance.

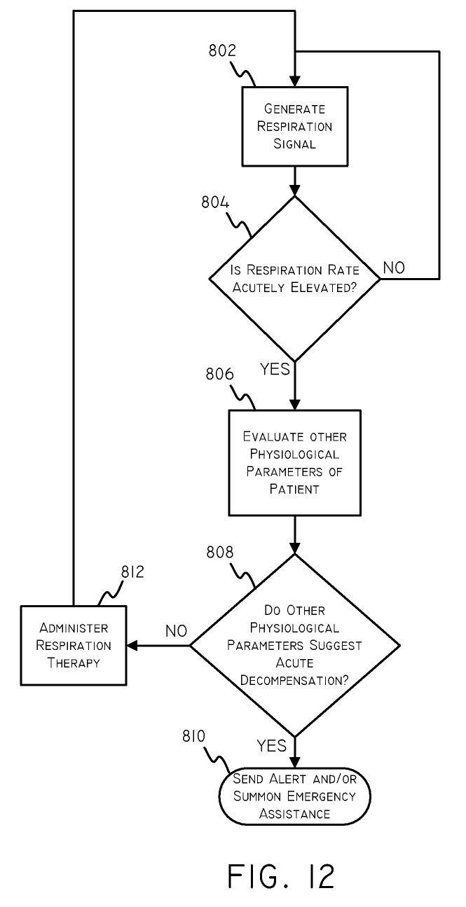

Referring now to FIG. 12, a flow chart is shown illustrating some aspects of a method in accordance with an embodiment of the invention. In operation 802, the system can generate a respiration signal. The respiration signal can be processed in order to derive information about a patient's breathing patterns, including the respiration rate. In operation 804, the system can evaluate whether the patient's respiration rate is acutely elevated. As used herein, “acutely elevated” shall refer to an elevation that occurs rapidly, such as over a period of time less than about 24 hours. This can be done in various ways, such as by determining whether the respiration rate has changed by at least a threshold value. If respiration rate is not acutely elevated, then the system can go back to operation 802. However, if respiration rate is acutely elevated, then the system can assess other physiological parameters in operation 806. By way of example, the system assess whether the patient is experiencing tachycardia, elevated venous pressure, changes in heart sounds, fluid retention, etc. In operation 808, the system can evaluate whether or not the other physiological parameters measured suggest that the patient is experiencing acute decompensation. If not, then the system can administer respiration therapy in operation 812 and then go back to operation 802. However, if the other physiological parameters suggest that the patient is experiencing acute decompensation that is not rectified by administering breathing therapy, then in operation 810 the system can send an alert notification and/or summon emergency assistance.

In some embodiments where the system includes a CRM device, if the system determines that the patient is likely suffering from acute decompensation, the system may initiate pacing therapy in addition to breathing therapy to ameliorate the decompensation event. For example, the system can initiate pacing of one or more chambers of the heart in order to establish an optimal cardiac rhythm in response to a decompensation event.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as “arranged”, “arranged and configured”, “constructed and arranged”, “constructed”, “manufactured and arranged”, and the like.

One of ordinary skill in the art will understand that the operations, circuitry, and methods shown and described herein with regard to various embodiments of the invention can be implemented using software, hardware, and combinations of software and hardware. As such, the illustrated and/or described operations, circuitry, and methods are intended to encompass software implementations, hardware implementations, and software and hardware implementations.

All publications and patent applications in this specification are indicative of the level of ordinary skill in the art to which this invention pertains. All publications and patent applications are herein incorporated by reference to the same extent as if each individual publication or patent application was specifically and individually indicated by reference.

This application is intended to cover adaptations or variations of the present subject matter. It is to be understood that the above description is intended to be illustrative, and not restrictive. The scope of the present subject matter should be determined with reference to the appended claims, along with the full scope of equivalents to which such claims are entitled.

What is claimed is:

1. A system for providing respiration therapy to a patient, comprising:
an implantable device comprising a chronically implanted respiration sensor, the respiration sensor configured to generate a signal indicative of respiration rate of the patient; and
an external interface device in communication with the implantable device, the external interface device comprising an output device and configured to deliver respiration therapy to the patient, the respiration therapy comprising one or more breathing prompts generated by the output device.

2. The system of claim 1, the respiration sensor selected from the group consisting of an impedance sensor, a pressure sensor, an accelerometer, and a blood gas concentration sensor.

3. The system of claim 1, the respiration sensor comprising a minute ventilation sensor.

4. The system of claim 1, the external interface device configured to be carried or worn by the patient.

5. The system of claim 1, the external interface device comprising a patient management system.

6. The system of claim 1, the external interface device comprising a handheld computing device.

7. The system of claim 1, the one or more breathing prompts comprising a video breathing prompt.

8. The system of claim 1, the one or more breathing prompts comprising an audio breathing prompt.

9. The system of claim 1, the one or more breathing prompts comprising a prompt for the patient to inhale and a prompt for the patient to exhale.

10. The system of claim 1, the implantable device further comprising a heart rate sensor.

11. The system of claim 10, the implantable device configured to generate breathing prompts timed to synchronize voluntary breathing with myocardial systole or diastole.

12. The system of claim 1, the system configured to monitor the patient's respiration rate over time and initiate respiration therapy when the respiration rate exceeds a threshold value.

13. The system of claim 1, further comprising an electrical stimulation lead comprising an electrode, the system configured to deliver electrical stimulation pulses to the phrenic nerve as breathing prompts.

14. A method for providing respiration therapy to a patient comprising:
transmitting a respiration signal of the patient from an implanted device to an external interface device; and
delivering respiration therapy to the patient via an external interface device; the respiration therapy comprising one or more prompts; wherein the prompts direct the patient to reduce their respiration rate to less than or equal to a target rate.

15. The method of claim 14, further comprising storing data regarding the patient's breathing and transmitting the data to a care provider.

16. The method of claim 14, wherein the system delivers respiration therapy in response to changes in the patient's breathing pattern exceeding predetermined values.

17. The method of claim 16, wherein the predetermined values are determined based on historical data regarding the patient's respiration rate.

18. The method of claim 14, further comprising storing and trending historical data regarding the patient's respiration signal.

19. The method of claim 16, further comprising summoning emergency assistance if the patient's respiration signal does not return to predetermined values after the system delivers respiration therapy.

20. The method of claim 16, the implanted device comprising a cardiac rhythm management device, further comprising the step of pacing the patient's heart if the patient's respiration signal does not return to predetermined values after the system delivers respiration therapy.

21. The method of claim 14, further comprising initiating respiration therapy upon patient request.

22. The method of claim 14, wherein the respiration signal is transmitted when a threshold change occurs in the respiration signal.

23. A method of monitoring a heart failure patient for decompensation events comprising:
generating a respiration signal with a chronically implanted respiration sensor;
monitoring the respiration signal for acute increases in respiration rate;
evaluating other physiological parameters of the patient; and
summoning emergency assistance if there is an acute increase in respiration rate and the other physiological parameters suggest an acute decompensation event is occurring.

24. An implantable system for providing respiration therapy to a patient, comprising:
an implantable device comprising a chronically implanted respiration sensor, the respiration sensor configured to generate a signal indicative of respiration rate of the patient;
an electrical stimulation lead comprising an electrode, the electrical stimulation lead in electrical communication with the implantable device;
the implantable device configured to administer respiration therapy to the patient, the respiration therapy comprising one or more breathing prompts, the breathing prompts comprising electrical stimulation pulses delivered to the phrenic nerve.
